(19) United States
(12) Patent Application Publication
(10) Pub. No.: US 2015/0267220 A1
(43) Pub. Date: Sep. 24, 2015

(54) Maize RING-H2 Genes and Methods of Use

(71) Applicant: PIONEER HI BRED INTERNATIONAL INC, Johnston, IA (US)

(72) Inventors: Norbert Brugiere, Johnston, IA (US); Jeffrey Habben, Urbandale, IA (US); Xiping Niu, Johnston, IA (US)

(21) Appl. No.: 14/351,084

(22) PCT Filed: Oct. 12, 2012
(86) PCT No.: PCT/US12/59882

§ 371 Date: Apr. 10, 2014

Related U.S. Application Data
(60) Provisional application No. 61/546,646, filed on Oct. 13, 2011.

(51) Int. Cl. C12N 15/82; C07K 14/415

(52) U.S. Cl. CPC C12N 15/8273; C07K 14/415

(57) ABSTRACT

The present invention relates to the field of plant molecular biology, more particularly to the regulation of genes that increase drought tolerance and yield. Provided herein are methods finding use in agriculture for increasing drought tolerance in dicot and monocot plants. Methods comprising introducing into a plant cell a polynucleotide that encodes a maize XERICO polypeptide operably linked to a promoter that drives expression in a plant are provided. Methods are further provided for maintaining or increasing yield in plants under drought conditions by introducing into a plant cell a polynucleotide encoding a maize XERICO poly-peptide and a polynucleotide encoding an abscisic acid (ABA)-associated polypeptide. Also provided are transformed plants, plant tissues, plant cells, and seeds thereof.

### FIELD

The present disclosure relates to the field of plant molecular biology, more particularly to the regulation of genes that increase drought tolerance and yield.

### BACKGROUND

Insufficient water for optimum growth and development of crop plants is a major obstacle to consistent or increased food production worldwide. Population growth, climate change, irrigation-induced soil salinity, and loss of productive agricultural land to development are among the factors contributing to a need for crop plants which can tolerate drought. Drought stress often results in reduced yield. In maize, this yield loss results in large part from plant failure to set and fill seed in the apical portion of the ear, a phenomenon known as tip kernel abortion.

Plants are restricted to their habitats and must adjust to the prevailing environmental conditions of their surroundings. To cope with abiotic stressors in their habitats, higher plants use a variety of adaptations and plasticity with respect to gene regulation, morphogenesis, and metabolism. Adaptation and defense strategies may involve the activation of genes encoding proteins important in the acclimation or defense towards different stressors including drought. Understanding and leveraging the mechanisms of abiotic stress tolerance will have a significant impact on crop productivity.

Methods are needed to enhance drought stress tolerance and to maintain or increase yield under drought conditions.

### SUMMARY

Methods are provided for increasing drought tolerance in plants. More particularly, the methods of the disclosure find use in agriculture for increasing drought tolerance in dicot and monocot plants. The methods comprise introducing into a plant cell a polynucleotide that encodes a maize XERICO polypeptide operably linked to a promoter that drives expression in a plant.

Methods are further provided for maintaining or increasing yield in plants under drought conditions. Certain embodiments comprise introducing into a plant cell a polynucleotide encoding a maize XERICO polypeptide and a polynucleotide encoding an abscisic acid (ABA)-associated polypeptide. Also provided are transformed plants, plant tissues, plant cells, and seeds thereof.

The following embodiments are among those encompassed by the present invention.
1. A method for increasing drought tolerance in a plant, said method comprising:
   a) introducing into said plant a polynucleotide construct comprising a nucleotide sequence encoding a polypeptide having at least 90% sequence identity to SEQ ID NO: 2 (ZmXERICO1), SEQ ID NO: 4 (ZmXERICO2), or SEQ ID NO: 6 (ZmXERICO1A), wherein said nucleotide sequence is operably linked to a heterologous promoter selected from the group consisting of a weak constitutive promoter, an organ- or tissue-preferred promoter (for example a root-specific promoter), a stress-inducible promoter, a chemical-induced promoter, a light-responsive promoter and a diurnally-regulated promoter.
   b) expressing said nucleotide sequence in said plant;
   whereby drought tolerance of said plant is increased relative to a control plant.
2. The method of embodiment 1, wherein said weak constitutive promoter is a GOS2 promoter or rice actin promoter.
3. The method of embodiment 1, wherein said tissue-preferred promoter is a leaf-preferred promoter, a root-preferred promoter, a vasculature-specific promoter or a promoter without expression in developing or mature ears.
4. The method of embodiment 1, wherein said stress-inducible promoter is a Rab17 promoter or an Rd29a promoter.
5. The method of embodiment 1, wherein said light-responsive promoter is an rbcS (ribulose-1,5-bisphosphate carboxylase) promoter, a Cab (chlorophyll a/b-binding) promoter or a phosphoenol-pyruvate carboxylase (PEPc) promoter.
6. The method of embodiment 1, wherein said diurnally-regulated promoter is disclosed in PCT/US2011/020314.
7. A method for increasing yield of a seed crop plant exposed to drought stress, said method comprising increasing expression of a polypeptide having at least 90% sequence identity to SEQ ID NO: 2, 4 or 6 in said plant and resulting in changed abscisic acid (ABA) homeostasis levels or decreasing responsiveness of developing seed of said plant to ABA.
8. The method of embodiment 7, wherein said crop plant further comprises an ABA-associated sequence operably linked to a heterologous promoter that drives expression in developing seed tissues.
9. The method of embodiment 8, wherein said ABA-associated sequence encodes an ABA-insensitive ABI mutant.
10. The method of embodiment 9, wherein said ABA-insensitive ABI mutant is selected from the group consisting of abi1, abi2 and ZmABI1 mutant.
11. The method of embodiment 7 or 8, wherein said seed crop plant is selected from the group consisting of a grain plant, an oil-seed plant, and a leguminous plant.
12. The method of embodiment 11, wherein said grain plant is corn or wheat.
13. The method of embodiment 11, wherein said oil-seed plant is a Brassica plant.
14. The method of any one of embodiments 7-13, wherein said promoter is an early kernel/embryo promoter.
15. The method of any one of embodiments 7-14, wherein a nucleotide sequence encoding said polypeptide is introduced into said plant by breeding or by transformation.
16. A plant comprising a polynucleotide construct comprising a nucleotide sequence encoding a polypeptide having at least 90% sequence identity to SEQ ID NO: 2, SEQ ID NO: 4 or SEQ ID NO: 6, wherein said nucleotide sequence is operably linked to a heterologous promoter selected from the group consisting of a weak constitutive promoter, an organ- or tissue-preferred promoter, a stress-inducible promoter, a chemical-induced promoter, a light-responsive promoter, and a diurnally-regulated promoter.
17. The plant of embodiment 16, wherein said polynucleotide is stably incorporated into the genome of said plant.
18. The plant of embodiment 16, wherein said plant is a seed crop plant.
19. The plant of embodiment 16, wherein said plant exhibits an increase in drought tolerance relative to a control plant.
20. A transformed seed of the plant of any one of embodiments 16-19.
21. A method of improving drought tolerance in a population of crop plants, the method comprising (a) expressing a recombinant protein comprising RING-H2 zinc finger motif, wherein the RING-H2 domain is present in one of SEQ ID NO: 2, SEQ ID NO: 4 or SEQ ID NO: 6; (b) exposing the crop plants to a drought condition in a field; and (c) improving the drought tolerance of the population of crop plants in the field.
22. A method of reducing phaseic acid (PA) and dihydrophaseic acid (DPA) levels in a plant, drought tolerance in a population of crop plants, the method comprising (a) expressing a recombinant protein comprising RING-H2 zinc finger motif, wherein the RING-H2 domain is present in one of SEQ ID NO: 2, SEQ ID NO: 4, or SEQ ID NO: 6; (b) exposing the crop plants to a drought condition in a field; and (c) reducing the phaseic acid (PA) and dihydrophaseic acid (DPA) levels in plant, while increasing the levels of ABA in the plant.

### BRIEF DESCRIPTION OF THE DRAWINGS

FIG. 1 presents sequence alignments of ZmXERICO proteins and AtXERICO. (A) Alignment of ZmXERICO1 (SEQ ID NO: 2) and ZmXERICO2 (SEQ ID NO: 4) with Arabidopsis Xerico (SEQ ID NO: 10). The first box (positions 13-35) indicates trans-membrane domain; the second box (positions 42-65) identifies Serine-rich domain of Xerico; and the third box (positions 106-147) identifies the RING-H2 domains. A consensus sequence is provided (SEQ ID NO: 7) (B) Identity and similarity table for XERICO proteins. Similarity scores are indicated in parentheses. Scores were calculated using the Needleman-Wunsch Algorithm with a gap creation penalty of 8 and a gap extension penalty of 2.

FIG. 2 presents graphs demonstrating relative fold expression levels of Xerico in the shoots and roots of 18-day Arabidopsis seedlings subjected to different abiotic stresses: cold (a), osmotic (b), salt (c), drought (d) and heat (e). Expression is presented as fold expression versus wild-type (untreated).

FIG. 3 presents graphs showing expression of ZmXERICO1 in corn roots and leaves under drought conditions, and in leaves in response to 24- and 48-hour abscisic acid (ABA) treatment.

FIG. 4 shows Northern data indicating that ZmXERICO1 is induced in shoot and root tissues when the plant is under drought stress. Rewatering of the plant removes the stress, and expression of ZmXERICO1 declines. The expression pattern of ZmXERICO2 is similar to ZmXERICO1 in roots; however, in shoots, ZmXERICO2 is expressed at low levels and is not induced by drought stress.

FIG. 5 presents a graph and Northern data depicting the fluctuating diurnal expression patterns of ZmXERICO1 in harvested maize samples. Peak expression was observed in leaves 2 hours after beginning of the dark period.

FIG. 6 is a series of graphs showing enhanced ABA sensitivity of plants over-expressing AtXerico or a Xerico homolog (Zm=maize) linked to the constitutive 35S promoter. ABA hypersensitivity is reflected in reduced germination percentages of transgenic plants compared to control plants.

FIG. 7 is a bar graph depicting levels of cis-abscisic acid and abscisic acid glucose ester in ZmXERICO transgenic plants and non-transgenic controls, shown as ng/g DW (dry weight). Far left bar of each set represents transgene-negative plants. Fifth bar of each set, counting from left, represents plants in which the transgenic event did not express. All other bars represent transgene-positive plants.

FIG. 8 is a series of bar graphs demonstrating that Ubi:ZmXERICO1 transgenic events have lower stomatal conductance and higher water use efficiency (WUE) relative to controls (“BN” and “WT”).

FIG. 9 is a graph depicting hypersensitivity to ABA, measured as root elongation rate in presence or absence of 50 μM ABA, of transgenic Ubi::ZmXERICO1 maize seedlings compared to bulk-null control plants. In each panel, Control-BN is on left; transgenic is on right.

FIG. 10 is a series of graphs showing that water loss during leaf dehydration is significantly reduced in Arabidopsis transgenic plants over-expressing ZmXERICO1 compared to controls.

### DETAILED DESCRIPTION

Methods are provided for increasing stress tolerance, particularly abiotic stress tolerance, in plants. These methods find use, for example, in increasing tolerance to drought stress and maintaining or increasing yield during drought conditions, particularly in agricultural plants. The methods involve genetically manipulating a plant to alter the expression of genes associated with the degradation, synthesis and/or perception of abscisic acid (ABA), a small, lipophilic plant hormone that modulates plant development, seed dormancy, germination, cell division and cellular responses to environmental stresses such as drought, cold, salt, pathogen attack, and UV radiation. See, for review, Chandler and Robinson, (1994) Annu. Rev. Plant Physiol. Plant Mol. Biol. 45:113-141; Rock, (2000) New Phytol. 148:357-396. In some embodiments, crop yield is maintained or increased by ameliorating the detrimental effects of ABA on seed or embryo development in agriculturally important plants.

The methods comprise stably incorporating into the genome of a plant a DNA construct comprising a nucleotide sequence which encodes a maize Xerico polypeptide, operably linked to a promoter that drives expression in a plant. Three maize Xerico polynucleotides and their encoded polypeptides are disclosed herein: ZmXERICO1, ZmXERICO2, and ZmXERICO1A. ZmXERICO1A is an allelic variant of ZmXERICO1; ZmXERICO1 and ZmXERICO1A polypeptides are over 98% identical. ZmXERICO1 and ZmXERICO2 polypeptides share approximately 83-88% sequence identity, depending on algorithm used. Maize Xerico polypeptides share approximately 32-35% amino acid sequence identity to Arabidopsis Xerico.

Xerico is a member of the RING (Really Interesting New Gene) zinc-finger protein superfamily. A RING finger domain is defined by the consensus sequence CX2CX(9-39)CX(1-3)HX(2-3)C/HX2CX(4-48)CX2C, where X is any amino acid and the number of X residues varies by RING polypeptide. Generally, RING finger proteins are enzymes that mediate the transfer of ubiquitin (Ub) to various substrates for proteolytic degradation. See, e.g., Freemont, (2000) Curr. Biol. 10:R84-87; Joazeiro and Weissman, Cell (2000) 102:549-52. Briefly, the ubiquitin pathway targets specific proteins for proteolysis by attaching Ub to the targeted protein using three enzymes, an activating enzyme (E1), a conjugating enzyme (E2), and the ubiquitin ligase (E3). See, for review, Stone and Callis, (2007) Plant Biol. 10:624-632.

Xerico is further characterized as comprising a RING-H2 zinc finger motif. Proteins comprising RING-H2 motifs, which are characterized by the presence of a histidine at the fifth coordination site (Liu, et al., (2008) Plant Cell 20:1538-1554), have been shown to have E3 ubiquitin ligase activity which facilitates the transfer of phosphorylated Ub to a heterologous substrate or to one of the polypeptide's own subunits as part of a regulated auto-ubiquitination process. See, e.g., Correia, et al., (2005) Annu. Rev. Pharmacol. Toxicol. 45:439-64.

While the invention is not bound by any particular theory or mechanism of action, it is believed that Xerico is a negative regulator of ABA degradation rather than a positive regulator of ABA synthesis. It is further believed that overexpression of Xerico promotes ubiquitin-mediated degradation of 8′-hydroxylases that catabolize ABA into the catabolites phaseic acid (PA) and diphaseic acid (DPA). See, Kushiro, et al., (2004) EMBO J. 23:1647-1656; Umezawa et al., (2006) Plant J. 46:171-182. Consistent with this model, it is believed that overexpression of Xerico will disrupt the delicate balance of ABA biosynthesis and catabolism by increasing degradation of 8′-hydroxylases and, in turn, promoting ABA accumulation in the plant.

In one aspect, methods are provided for increasing abiotic stress tolerance, such as drought tolerance, in a plant. In some embodiments, the methods can comprise introducing into a plant a polynucleotide construct comprising a nucleotide sequence encoding a polypeptide having at least about 90% amino acid sequence identity to SEQ ID NO: 2, SEQ ID NO: 4 or SEQ ID NO: 6 or a variant or fragment thereof, operably linked to a heterologous promoter that is functional in a plant cell. In certain embodiments, when a nucleotide sequence provided herein is expressed in the plant, drought tolerance of the plant is increased relative to a control plant. In some cases, the nucleotide sequence encodes a polypeptide having at least about 70%, about 75%, about 80%, about 85%, about 90%, about 95%, about 97%, about 99% or about 100% amino acid sequence identity to SEQ ID NO: 2, SEQ ID NO: 4, or SEQ ID NO: 6 or a variant or fragment thereof. In some cases, the nucleotide sequence encodes SEQ ID NO: 2, SEQ ID NO: 4 or SEQ ID NO: 6.

Xerico polypeptides disclosed herein can be altered in various ways including amino acid substitutions, deletions, truncations, and insertions. Methods for such manipulations are generally known in the art. For example, sequence variants of the Xerico polypeptides can be prepared by mutations in the DNA encoding each. Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. A mutagenic and recombinogenic procedure such as DNA shuffling can be employed to alter the Xerico polypeptides disclosed herein. Thus, the genes and nucleotide sequences of the invention involve both the naturally occurring sequences as well as mutant forms. Likewise, the proteins of the invention encompass naturally occurring polypeptides as well as variations and modified forms thereof. Such variants will continue to possess the desired functional activity. In that regard, mutations that will be made in the DNA encoding the variant must not place the sequence out of reading frame and preferably will not create complementary regions that could produce secondary mRNA structure. See, EP Patent Application Publication Number 75,444.

Accordingly, the present disclosure encompasses the maize Xerico polypeptides as well as active variants and fragments thereof. That is, it is recognized that variants and fragments of the proteins may be produced that retain the ability to increase ABA levels in a plant. Such variants and fragments include truncated sequences as well as N-terminal, C-terminal, and internally-deleted amino acid sequences of the proteins. By “fragment” is intended a portion of the polynucleotide or a portion of the amino acid sequence and hence of the protein encoded thereby. Fragments of a polynucleotide may encode protein fragments that retain biological activity and hence retain the ability to increase ABA accumulation in a plant. Alternatively, fragments of a polynucleotide which are useful as hybridization probes generally do not encode fragment proteins retaining biological activity. Thus, fragments of a nucleotide sequence may range from at least about 20 nucleotides to about 50 nucleotides, about 100 nucleotides and up to the full-length polynucleotide encoding a maize Xerico protein.

A fragment of a polynucleotide that encodes a biologically active portion of a claimed Xerico protein will encode at least about 15, about 25, about 30, about 50, about 100 or about 150 contiguous amino acids, or up to the total number of amino acids present in a full-length Xerico protein of the disclosure (for example, 157 amino acids for SEQ ID NO: 2, 165 amino acids for SEQ ID NO: 4, and 155 for SEQ ID NO: 6, respectively). Fragments of a polynucleotide which are useful as hybridization probes or PCR primers generally need not encode a biologically active portion of Xerico protein. Thus, a fragment of a polynucleotide may encode a biologically active portion of a Xerico protein, or it may be a fragment that can be used as a hybridization probe or PCR primer using methods disclosed below. A biologically active portion of a Xerico protein can be prepared by isolating a portion of a Xerico polynucleotide, expressing the encoded portion of the Xerico protein (e.g., by recombinant expression in vitro), and assessing the activity of the encoded portion of the Xerico protein. Polynucleotides that are fragments of a Xerico nucleotide sequence comprise at least about 75, about 100, about 150, about 200, about 250, about 300, about 350, about 400, about 450 or about 470 contiguous nucleotides, or up to the number of nucleotides present in a full-length Xerico polynucleotide disclosed herein (for example, 474, 498, and 465 nucleotides for SEQ ID NOS: 1, 3 and 5, respectively).

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a Xerico polypeptide disclosed herein. Variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a Xerico protein disclosed. Generally, variants of a particular polynucleotide will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein.

Variants of a particular reference polynucleotide disclosed can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 2, SEQ ID NO: 4 or SEQ ID NO: 6 is disclosed. Percent sequence identity between any two polypeptides can be calculated using sequence alignment programs and parameters described elsewhere herein. Where any given pair of polynucleotides is evaluated by comparison of the percent sequence identity shared by the two polypeptides they encode, the percent sequence identity between the two encoded polypeptides is at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention may be biologically active; that is, they continue to possess the desired biological activity of the native protein, that is, the ability to increase ABA accumulation in a plant as described herein. Such variants may result from, for example, genetic polymorphism or from human manipulation. Biologically active variants of a native Xerico protein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs and parameters described elsewhere herein. A biologically active variant of a reference protein may differ from that protein by as few as 1-15 amino acid residues, as few as 1-10, such as 6-10, as few as 5, as few as 4, 3, 2 or even 1 amino acid residue.

In certain embodiments, disclosed proteins may be altered in various ways including amino acid substitutions, deletions, truncations, and insertions. Methods for such manipulations are generally known in the art. For example, amino acid sequence variants and fragments of the Xerico proteins can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions and substitutions of the protein sequences encompassed herein are not expected to produce radical changes in the characteristics of the protein. When it is difficult, however, to predict the exact effect of a substitution, deletion or insertion in advance of making such modifications, one skilled in the art will appreciate that the effect will be evaluated by routine screening assays. That is, changes in ABA levels can be evaluated by standard methods known to those of ordinary skill in the art. Conventional methods for measuring ABA include, without limitation, antibody and enzyme-linked immunosorbent assays (ELISA), high-performance liquid chromatography (HPLC), gas chromatography/mass spectrometry (MS), and liquid chromatography/tandem mass spectrometry methods.

The following terms are used to describe the sequence relationships between two or more polynucleotides or polypeptides: (a) “reference sequence”, (b) “comparison window”, (c) “sequence identity” and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990), supra. BLAST nucleotide searches can be performed with the BLASTN program, score=100, wordlength=12, to obtain nucleotide sequences homologous to a nucleotide sequence encoding a Xerico protein. BLAST protein searches can be performed with the BLASTX program, score=50, wordlength=3, to obtain amino acid sequences homologous to a Xerico protein or polypeptide. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997), supra. When utilizing BLAST, Gapped BLAST, PSI-BLAST, the default parameters of the respective programs (e.g., BLASTN for nucleotide sequences, BLASTX for proteins) can be used. See, www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3 and the nwsgapdna.cmp scoring matrix; % identity and % similarity for an amino acid sequence using GAP Weight of 8 and Length Weight of 2 and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As described herein, a nucleotide sequence encoding a Xerico polypeptide, variant or fragment thereof as provided herein is operably linked to a promoter that drives expression of the sequence in a plant. Any one of a variety of promoters can be used with a Xerico sequence, depending on the desired timing and location of expression. In some cases, the promoter is a constitutive promoter, a tissue-preferred promoter, a chemical-inducible promoter, a stress-inducible promoter, a light-responsive promoter or a diurnally-regulated promoter. For example, constitutive promoters can be used to drive expression of a nucleotide sequence of interest. The most common promoters used for constitutive overexpression are derived from plant virus sources, such as the cauliflower mosaic virus (CaMV) 35S promoter (Odell, et al., (1985) Nature 313:810-812). The CaMV 35S promoter delivers high expression in virtually all regions of transgenic monocot and dicot plants. Constitutive promoters also can include, for example, the core promoter of the Rsyn7 promoter and other constitutive promoters disclosed in WO 1999/43838 and U.S. Pat. No. 6,072,050; rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611.

Transgene expression can be beneficially adjusted by using a promoter suitable for the plant's background and/or for the type of transgene. Where low level expression is desired, weak promoters can be used. It is recognized that weak constitutive, weak inducible, or weak tissue-preferred promoters can be used. Generally, by “weak promoter” is intended a promoter that drives expression of a coding sequence at a low level. By low level is intended at levels of about 1/1000 transcripts to about 1/100,000 transcripts to about 1/500,000 transcripts. An example of a weak constitutive promoter is the GOS2 promoter; see, U.S. Pat. No. 6,504,083. While the claims are not bound by any particular theory or mechanism of action, it is believed that a significant but not excessive increase in ABA levels resulting from a low level of Xerico overexpression would promote drought tolerance in the plant without significant negative effects on yield.

In some embodiments, the Xerico sequences can be utilized with tissue-preferred or developmental-preferred promoters to drive expression of the sequence of interest in a tissue-preferred or a developmentally-preferred manner. For example, tissue-preferred promoters such as leaf-preferred promoter or root-preferred promoters can be used. While the claims are not bound by any particular theory or mechanism of action, it is believed that expression of Xerico in a root-preferred or leaf-preferred manner would promote drought tolerance in the plant without a significant detrimental impact on plant yield.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Root-preferred promoters are also known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. Leach and Aoyagi, (1991) describe their analysis of the promoters of the highly expressed roIC and roID root-inducing genes of Agrobacterium rhizogenes (see, Plant Science (Limerick) 79(1):69-76). Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene, fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772); and roIB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179. Other root-preferred promoters include ZmNAS2 promoter (U.S. Pat. No. 7,960,613), ZmCyclo1 promoter (U.S. Pat. No. 7,268,226), ZmMetallothionein promoters (U.S. Pat. Nos. 6,774,282; 7,214,854 and 7,214,855 (also known as RootMET2)), ZmMSY promoter (US Patent Application Publication Number 2009/0077691), Sb RCC3 promoter (US Patent Application Publication Number 2012/0210463) or MsZRP promoter (U.S. Pat. No. 5,633,363).

Other promoters may be utilized to drive expression of a maize Xerico polynucleotide, such as the promoter of the maize KZM2 gene (see, Buchsenschutz, et al., (2005) Planta 222:968-976 and NCBI Accession Number AY919830) or a green-tissue-preferred promoter (US Patent Application Publication Number 2011/0209242).

Constructs may also include one or more of the CaMV35S enhancer, Odell, et al., (1988) Plant Mol. Biol. 10:263-272, the ADH1 INTRON1 (Callis, et al., (1987) Genes and Dev. 1:1183-1200), the UBI1ZM INTRON (PHI) as an enhancer, and PINII terminator.

In some embodiments, the Xerico sequences can be utilized with stress-inducible promoters to drive expression of the sequence of interest in a stress-regulated manner. A stress-inducible promoter can be, for example, a rab17 promoter (Vilardell, et al., (1991) Plant Molecular Biology 17(5):985-993; Busk, et al., (1997) Plant J 11(6):1285-1295) or rd29a promoter (Yamaguchi-Shinozaki and Shinozaki, (1993) Mol. Gen. Genet. 236:331-340; Yamaguchi-Shinozaki and Shinozaki, (1994) Plant Cell 6:251-264). It has been shown that conditional inactivation of ERA1, a negative regulator of the ABA guard cell response, by expressing an era1 RNAi construct under control of a stress-induced rd29a promoter, improved canola plant tolerance to drought without a decrease in yield under well-watered conditions (Wang, et al., (2005) Plant J. 43:413-424). Thus, while the claims are not bound by any particular theory or mechanism of action, it is believed that overexpression of Xerico under control of a stress-induced promoter would promote increased drought stress tolerance without a significant concomitant decrease in plant yield. In addition, expression driven by a guard cell promoter such as is disclosed in U.S. Provisional Patent Application Ser. No. 61/712,301, filed Oct. 11, 2012, incorporated herein by reference.

Light-inducible and/or diurnally-regulated promoters can be used to drive expression of a nucleotide sequence in a light-dependent manner. A light-responsive promoter can be, for example, a rbcS (ribulose-1,5-bisphosphate carboxylase) promoter which responds to light by inducing expression of an associated gene. In some cases, diurnally-regulated promoters can be used to drive expression of a nucleotide sequence in a manner regulated by light and/or the circadian clock. For example, a cab (chlorophyll a/b-binding) promoter can be used to produce diurnal oscillations in gene transcription. In some embodiments, a diurnally-regulated promoter can be a promoter region as disclosed in U.S. patent application Ser. No. 12/985,413, herein incorporated by reference. In some embodiments, a promoter can be used that drives expression of a nucleotide sequence in a diurnally-regulated manner but further with a temporal expression pattern opposite of that of endogenous ZmXERICO1 or ZmXERICO2.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Parameters such as gene expression level, water use efficiency, ABA sensitivity, and others are typically presented with reference to a control cell or control plant. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null.

In another aspect, the present invention also provides methods for maintaining or increasing yield of a seed crop plant exposed to drought stress, where the methods include increasing expression of a polypeptide having at least 90% sequence identity to SEQ ID NO: 2, SEQ ID NO: 4, or SEQ ID NO: 6 or a variant or fragment thereof, in the plant while also decreasing responsiveness of developing seed of the plant to the resulting accumulation of ABA. For example, methods can further comprise introducing into a target plant certain sequences that modulate ABA perception and/or signal transduction. In particular, it may be advantageous to introduce into a target plant sequences that modulate ABA perception and signal transduction in certain tissues such as, for example, tissues associated with seed initiation or development. By “sequences that modulate ABA perception and/or signal transduction” is intended genes and their mutant forms that disrupt biosynthesis and catabolism of ABA or its perception and/or signal transduction. These mutants, genes, and sequences that disrupt ABA synthesis or its perception and/or signal transduction are also called “ABA-associated sequences” herein. An ABA-associated sequence can further be as disclosed in US Patent Application Publication Number 2004/0148654, which is herein incorporated by reference. Such sequences include, without limitation, ABA-insensitive and hypersensitive mutants having altered sensitivity to ABA, or antisense sequences corresponding to the mutant or wild-type genes. ABA mutants are known in the art and include abi1-5, era1-3 (Cutler, et al., (1996) Science 273:1239-41), gca1/8 (Benning, et al., (1996) Proc. Workshop Abscisic Acid Signal Transduction in Arabidopsis, Madrid, p. 34), axr2 (Wilson, et al., (1990) Mol. Gen. Genet. 222:377-83), jar1 (Staswick, et al., (1992) Proc. Natl. Acad. Sci. USA 89:6837-40), jin4 (Berger, et al., (1996) Plant Physiol. 111:525-31), bri1 (Clouse, et al., (1996) Plant Physiol. 111:671-78) (Hordeum vulgare); aba1 (Bitoun, et al., (1990) Mol. Gen. Genet. 220:234-39 and Leydecker, et al., (1995) Plant Physiol. 107:1427-31) (Nicotiana plumbaginifolia); and the like. These and other ABA-associated mutants can be used in the practice of the invention.

Arabidopsis ABA-insensitive, ABI, mutants are available. Such mutants have pleiotropic effects in seed development, including decreased sensitivity to ABA inhibition of germination in altered seed-specific gene expression. See, Finkelstein, et al., (1998) The Plant Cell 10:1043-1045; Leung, et al., (1994) Science 264:1448-1452; Leung, (1997) Plant Cell 9:759-771; Giraudat, et al., (1992) Plant Cell 14:1251-1261; Myer, et al., (1994) Science 264:1452-1455; Koornneef, et al., (1989) Plant Physiol. 90:463-469; Nambara, et al., (1992) Plant J. 2:435-441; Finkelstein and Somerville, (1990) Plant Physiol. 94:1172-1179; Leung and Giraudat, (1998) Annu. Rev. Plant Physiol. Plant Mol. Biol. 49:199-222; Robinson and Hill, (1999) Plant, Cell and Environment 22:117-123 and Rodriguez, et al., (1998) FEBS Letters 421:185-190 and the references cited therein, all of which are herein incorporated by reference. Other ABA-associated mutants include bri1 from Arabidopsis thaliana, the sequence of which can be found in Genbank Accession Number AF017056 and Li, et al., (1997) Cell 90:929-938, both of which are herein incorporated by reference. A further ABA-associated mutant is ZmABI1 (SEQ ID NOS: 8 and 9), which is a maize ABA-associated mutant that is similar to the Arabidopsis G180D mutant and which was disclosed as SEQ ID NOS: 11-12 in US Patent Application Publication Number 2009/0205067, which is herein incorporated by reference.

An abi mutant of interest includes, for example, Arabidopsis abi1, a dominant mutation in the structural part of the ABM gene, which encodes a protein phosphatase 2C (PP2C). This mutation comprises a nucleic base transition from guanine to adenine which changes the DNA sequence GGC to GAC, thus causing the wild type glycine residue at amino acid position 180 to be replaced with aspartic acid (referred to as G180D; Meyer, et al., (1994) Science 264:1452-1455).

Certain embodiments of the invention utilize the ABA-associated sequences described herein to control the plant response to ABA. Generally, it will be beneficial to block ABA perception or hypersensitivity in selected tissues, such as female reproductive tissues, to prevent a loss of yield. Utilizing the ABA-associated sequences, coding sequences, and antisense sequences, the expression and perception of ABA in a plant can be controlled. Such sequences can be introduced into plants of interest by recombinant methods as well as by traditional breeding methods.

For the expression of a polynucleotide construct comprising an ABA-associated sequence in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 days after pollination (“DAP”). During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, cim1, a promoter that is active 5 DAP in particular tissues. See, for example, WO 2000/11177, which is herein incorporated by reference. Other early kernel/embryo promoters include the seed-preferred promoters end1, which is active 7-10 DAP and end2, which is active 9-14 DAP in the whole kernel and active 10 DAP in the endosperm and pericarp. See, for example, WO 2000/12733, herein incorporated by reference. Additional early kernel/embryo promoters that find use in certain methods of the present invention include the seed-preferred promoter Itp2, U.S. Pat. No. 5,525,716; maize Zm40 promoter, U.S. Pat. No. 6,403,862; maize nuc1c, U.S. Pat. No. 6,407,315; maize ckx1-2 promoter, U.S. Pat. No. 6,921,815 and US Patent Application Publication Number 2006/0037103; maize lec1 promoter, U.S. Pat. No. 7,122,658; maize ESR promoter, U.S. Pat. No. 7,276,596; maize ZAP promoter, US Patent Application Publication Numbers 2004/0025206 and 2007/0136891; maize promoter eep1, US Patent Application Publication Number 2007/0169226 and maize promoter ADF4, U.S. Patent Application Ser. No. 60/963,878, filed Aug. 7, 2007. These promoters drive expression in developing seed tissues.

Such early kernel/embryo promoters can be used with genes or mutants in the perception/signal transduction pathway for ABA. In this manner, mutant genes such as abi1 or abi2 operably linked to an early kernel/embryo promoter would dominantly disrupt ABA action in the targeted tissues but not alter the later required ABA function in seed maturation. Alternatively, an early kernel/embryo promoter can be operably linked to a wild type (co-suppression) or antisense nucleotide sequence of an ABA associated sequence. The early kernel/embryo promoter would be utilized to disrupt ABA action in certain tissue prior to seed maturation.

Nucleotide sequences encoding maize Xerico polypeptides and/or other polynucleotides of the present invention can be introduced into a plant. The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, breeding methods, stable transformation methods, transient transformation methods, and virus-mediated methods. “Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell targeted for transformation. For example, different methods may be preferred for use in monocots or in dicots. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722) and ballistic particle acceleration (see, for example, U.S. Pat. No. 4,945,050; U.S. Pat. No. 5,879,918; U.S. Pat. Nos. 5,886,244 and 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926); and Lec1 transformation (WO 2000/28058). See also, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens); all of which are herein incorporated by reference.

In specific embodiments, polynucleotide sequences of the invention can be provided to a plant using any of a variety of transient transformation methods. Such transient transformation methods include, but are not limited to, the introduction of the Xerico protein or variants and fragments thereof directly into the plant or the introduction of the Xerico transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the invention can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

In some cases, it is convenient to introduce nucleotide sequences of the invention as expression cassettes. Such expression cassettes can comprise 5′ and 3′ regulatory sequence operably linked to a Xerico polynucleotide of the invention or ABA-associated polynucleotide of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein-coding regions, contiguous and in the same reading frame. The expression cassette may additionally contain at least one additional gene to be cotransformed into the organism. Alternatively, additional gene(s) can be provided on multiple expression cassettes. Expression cassettes can be provided with a plurality of restriction sites for insertion of the gene of interest to be under the transcriptional regulation of the regulatory regions. The expression cassette may additionally contain selectable marker sequences.

In some embodiments, an expression cassette will include in the 5′-3′ direction of transcription, a transcriptional and translational initiation region (i.e., a promoter), a Xerico polynucleotide of the invention, and a transcriptional and translational termination region (i.e., termination region) functional in plants. The regulatory regions (i.e., promoters, transcriptional regulatory regions, and translational termination regions) and/or the Xerico polynucleotide of the invention may be native/analogous to the host cell or to each other. Alternatively, the regulatory regions and/or the Xerico polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

While it may be optimal to express the sequences using heterologous promoters, the native promoter sequences may be used. Such constructs can change expression levels of Xerico in the plant or plant cell. Thus, the phenotype of the plant or plant cell can be altered.

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” meganucleases produced to modify plant genomes (see, e.g., WO 2009/114321; Gao, et al., (2010) Plant Journal 1:176-187). Another site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41. A transcription activator-like (TAL) effector-DNA modifying enzyme (TALE or TALEN) is also used to engineer changes in plant genome. See e.g., US Patent Application Publication Number 2011/0145940, Cermak, et al., (2011) Nucleic Acids Res. 39(12) and Boch, et al., (2009) Science 326(5959):1509-12.

The termination region may be native with the transcriptional initiation region, may be native with the operably linked Xerico polynucleotide of interest, may be native with the plant host, or may be derived from another source (i.e., foreign or heterologous) to the promoter, the Xerico polynucleotide of interest, the plant host or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gown, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference. The plant preferred codons may be determined from the codons of highest frequency in the proteins expressed in a monocot or dicot of interest. Likewise, the optimized sequence can be constructed using monocot-preferred or dicot-preferred codons. See, for example, Murray, et al., (1989) Nucleic Acids Res. 17:477-498. It is recognized that all or any part of the gene sequence may be optimized or synthetic. That is, fully optimized or partially optimized sequences may also be used.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20) and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments; other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The maize Xerico polypeptides described herein may be used alone or in combination with additional polypeptides or agents to increase drought stress tolerance in plants. For example, in the practice of certain embodiments, a plant can be genetically manipulated to produce more than one polypeptide associated with increased drought tolerance. Those of ordinary skill in the art realize that this can be accomplished in any of a number of ways. For example, each of the respective coding sequences for polypeptides described herein can be operably linked to a promoter and then joined together in a single continuous DNA fragment comprising a multigenic expression cassette. Such a multigenic expression cassette can be used to transform a plant to produce the desired outcome. Alternatively, separate plants can be transformed with expression cassettes containing one or a subset of the desired coding sequences. Transformed plants that exhibit the desired genotype and/or phenotype can be selected by standard methods available in the art such as, for example, immunoblotting using antibodies which bind to the proteins of interest, assaying for the products of a reporter gene, and the like. Then, all of the desired coding sequences can be brought together into a single plant through one or more rounds of cross-pollination utilizing the previously selected transformed plants as parents.

Methods for cross-pollinating plants are well known to those skilled in the art, and are generally accomplished by allowing the pollen of one plant, the pollen donor, to pollinate a flower of a second plant, the pollen recipient, and then allowing the fertilized embryos in the pollinated flower to mature into seeds. Progeny containing the entire complement of desired coding sequences of the two parental plants can be selected from all of the progeny by standard methods available in the art as described supra for selecting transformed plants. If necessary, the selected progeny can be used as either the pollen donor or pollen recipient in a subsequent cross-pollination. Selfing of appropriate progeny can produce plants that are homozygous for both added, heterologous genes. Back-crossing to a parental plant and out-crossing with a non-transgenic plant are also contemplated, as is vegetative propagation. Descriptions of other breeding methods that are commonly used for different traits and crop plants can be found in several references, e.g., Fehr, (1987) Breeding Methods for Cultivar Development, ed. J. Wilcox (American Society of Agronomy, Madison, Wis.).

Compositions and methods disclosed herein may be used for transformation of any plant species, including, but not limited to, monocots and dicots. In some cases, plant species useful in the methods provided herein can be seed crop plants such as grain plants, oil-seed plants, and leguminous plants. Of particular interest are plants where the seed is produced in high amounts, or the seed or a seed part is edible. Seeds of interest include the grain seeds such as wheat, barley, rice, corn (maize), rye, millet and sorghum. Plants of particular interest are corn, wheat and rice.

Examples of plant species of interest include, but are not limited to, corn (maize; Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus effiotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The article “a” and “an” are used herein to refer to one or more than one (i.e., to at least one) of the grammatical object of the article. By way of example, “an element” means one or more element.

The following examples are presented by way of illustration, and not by way of limitation.

### EXPERIMENTAL

### Example 1

### Characteristics of ZmXERICO

Sequence Analysis

Two ZmXERICO genes have been identified and designated ZmXERICO1 (SEQ ID NO: 1), ZmXERICO2 (SEQ ID NO: 3) and ZmXERICO1A (SEQ ID NO: 5). Using Orthofind, several homologs to ZmXERICO genes from other species were identified, including those from sorghum and rice (Table 1).

TABLE 1
Summary of Orthofind Results Using ZmXERICO genes

| | | |
|---|---|---|
| Species | Gene ID | Relation |

ZmXERICO1 (PCO595065)

| | | |
|---|---|---|
| Arabidopsis thaliana | At2g04240.1 | candidate |
| | At1g15100.1 | family |
| Glycine max | Glyma18g01720.1 | ortholog |
| | Glyma13g11570.1 | ortholog |
| Oryza sativa | LOC_Os08g38460.1 | ortholog |
| | LOC_Os09g30160.1 | family |
| | LOC_Os02g45710.1 | family |
| Sorghum bicolor | Sb02g027680.1 | subtree neighbor |
| Zea mays | pco626546 | ultra-paralog |

ZmXERICO2 (PCO632835)

| | | |
|---|---|---|
| Arabidopsis thaliana | At2g04240.1 | ortholog |
| Glycine max | Glyma13g11570.1 | ortholog |
| | Glyma13g11570.1 | ortholog |
| Oryza sativa | LOC_Os08g38460.1 | candidate |
| | LOC_Os09g30160.1 | family |
| | LOC_Os01g16120.1 | family |
| Sorghum bicolor | Sb02g027680.1 | ortholog |

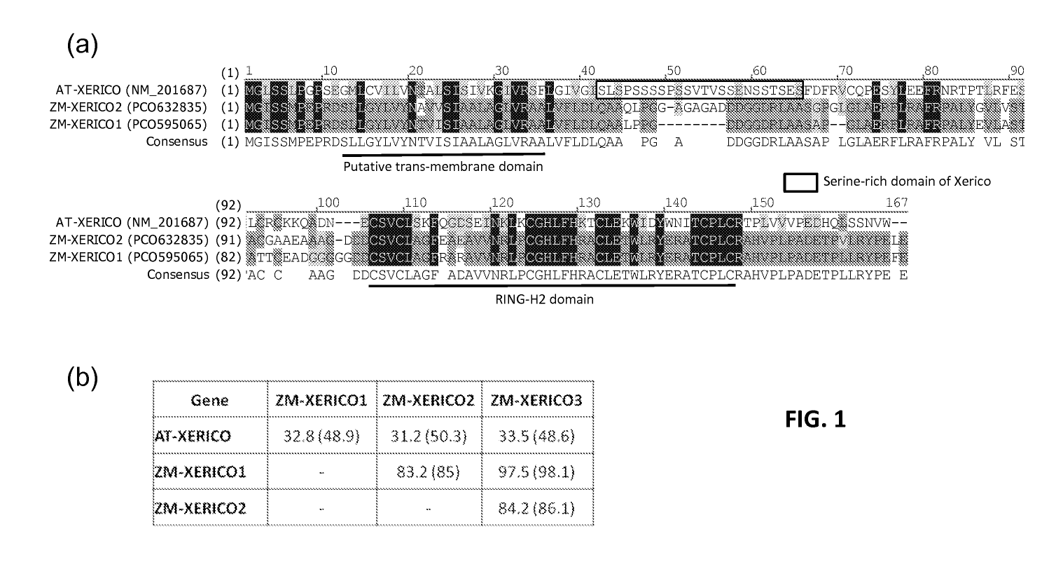

An alignment of maize (ZmXERICO) with Arabidopsis (AtXERICO) Xerico proteins showed low amino acid conservation, with overall identity scores ranging from 31 to 33% over 160 amino acids (FIG. 1). ZmXERICO1A differs from ZmXERICO1 by only four amino acids: Arginine (R) to Glutamine (Q) at position 54, Alanine (A) to Glycine (G) at position 59 and a deletion of 2 Glycines at position 91.

Analysis of the protein sequences using InterProScan identified a putative transmembrane region from amino acid residue 13 to amino acid residue 35 as well as a RING-H2 domain in the ZmXERICO proteins which overlaps the transmembrane region of Xerico. Unlike the Arabidopsis protein, maize Xerico proteins do not present a Serine-rich domain.

Characterization of ZM-XERICO Gene Expression

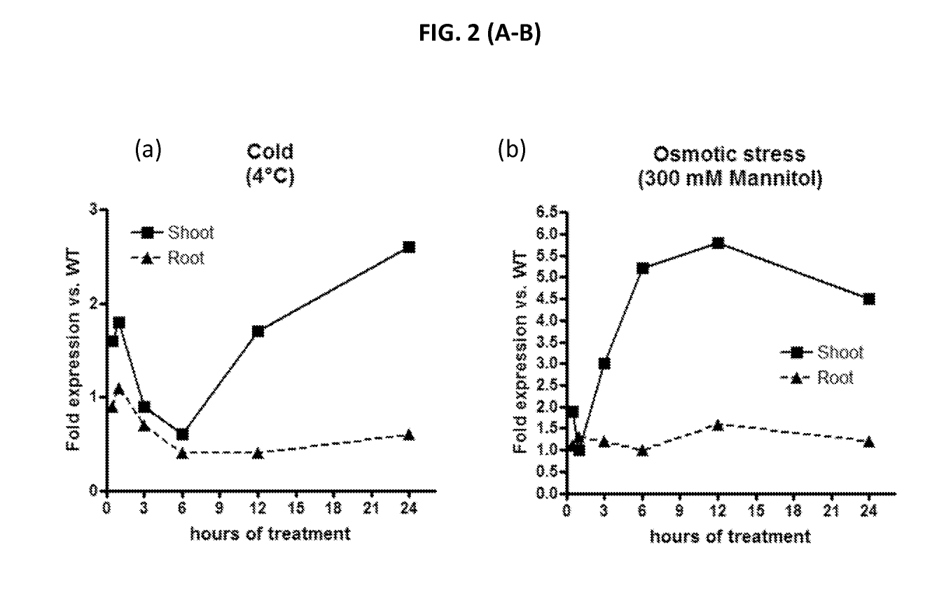

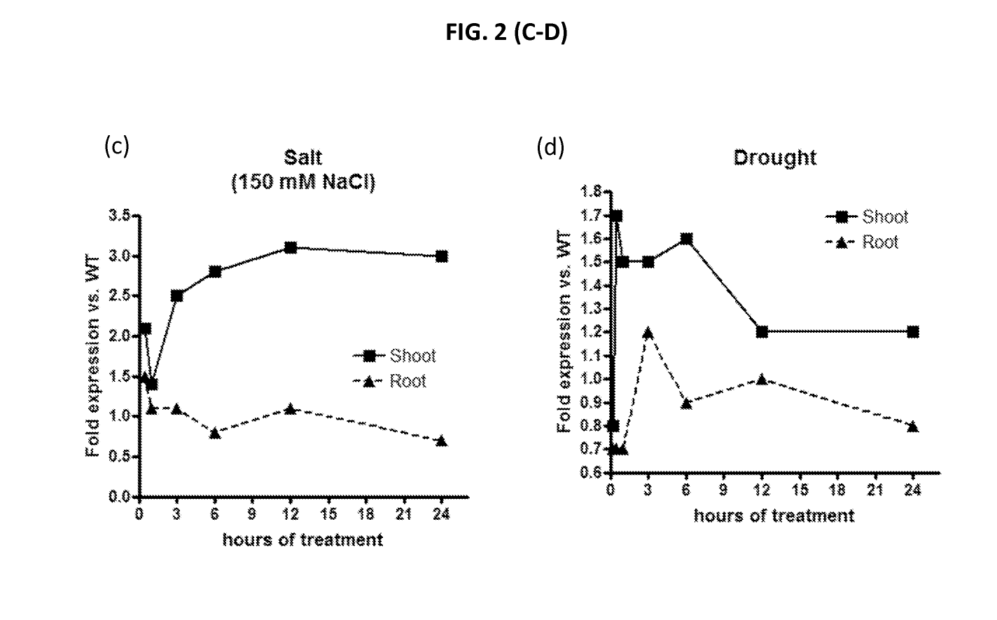

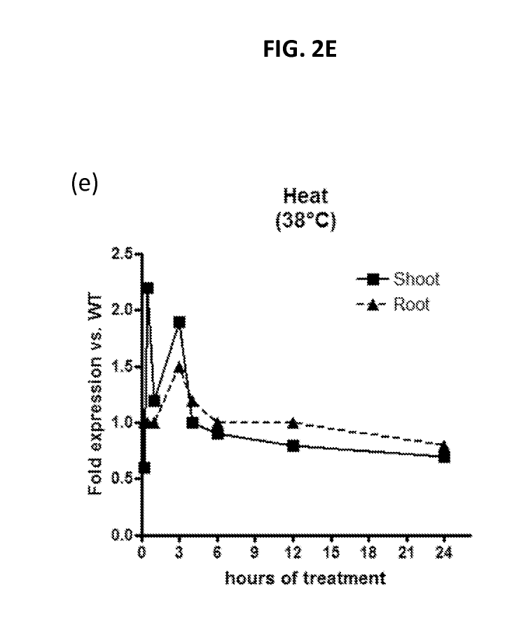

When seedling expression levels from publicly-available data (see, Microarray data from AtGenExpress of The Arabidopsis Information Resource) were further analyzed, this induction seemed to be somewhat stronger in shoot than root (FIG. 2). No dramatic induction of gene expression by ABA could be seen in publicly available data, but only three time points and 10 μM ABA treatments were available in the art.

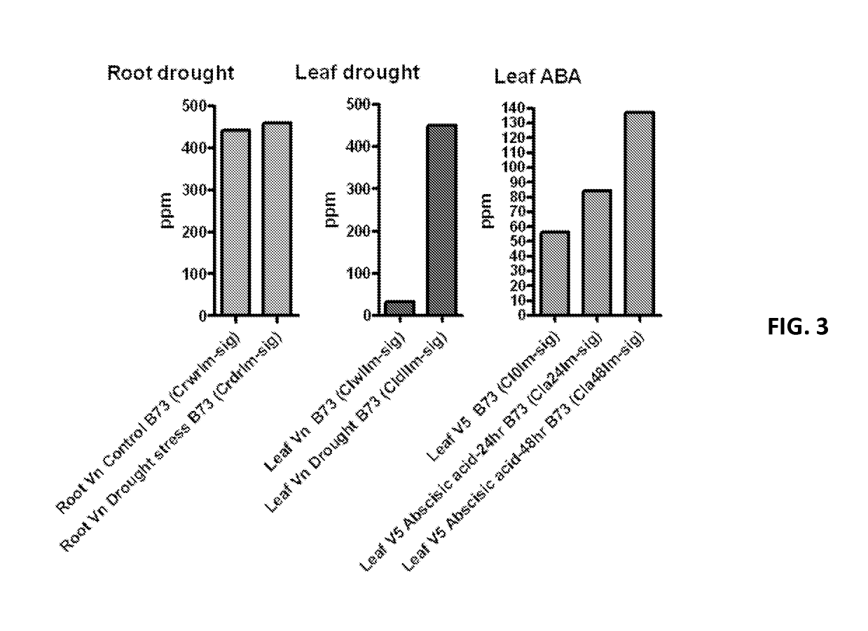

Similar to the Arabidopsis gene, expression of ZmXERICO is induced by drought in leaves but appears not induced in roots using a proprietary electronic expression database (FIG. 3). Expression levels were slightly increased in leaves at 24 hours (1.5×) and 48 hours (2×) after treatment by ABA (FIG. 3). The fact that expression appeared not to be inducible by drought in roots, the site where plants would perceive stress first, indicated that the site of action of ZmXERICO is organ specific and that use of tissue/organ specific promoters is an area for optimization of this lead.

The ZmXERICO1 expression pattern was studied using Lynx MPSS viewer. The gene is expressed in most corn tissues at levels averaging a few hundred parts per million (ppm). The maximum expression level was found in pericarp (R4) with 915 ppm and in stalk vascular bundles (V10-V11) with 1013 ppm.

Another interesting expression pattern for ZmXERICO1 can be found in immature ears with an increasing gradient of expression from the base of the ear (155 ppm) to the tip (886 ppm). A possible weak induction by nitrate has been reported, indicating an ABA response because there is evidence that ABA plays a role in mediating the regulatory effects of nitrate for example on root-branching (Signoram et al., (2001) Plant J. 28:655-662) or nodulation and nitrogen fixation in legumes (Tominaga, et al., (2009) Plant Physiol. 151:1965-76).

Diurnal Expression of ZmXERICO1

Diurnal expression data suggest that ZmXERICO expression is low during the day under normal growing conditions but is induced to higher levels at night. These findings were confirmed independently using Northern blot. It may be useful to express ZmXERICO under control of a diurnally-regulated promoter which increases expression during the day, when drought stress may be most severe.

Drought Induction of ZM-XERICO Gene Expression

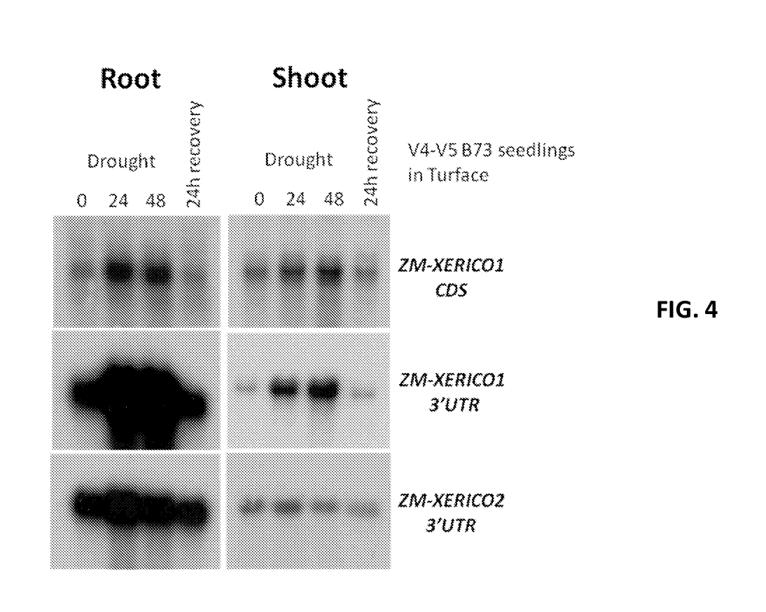

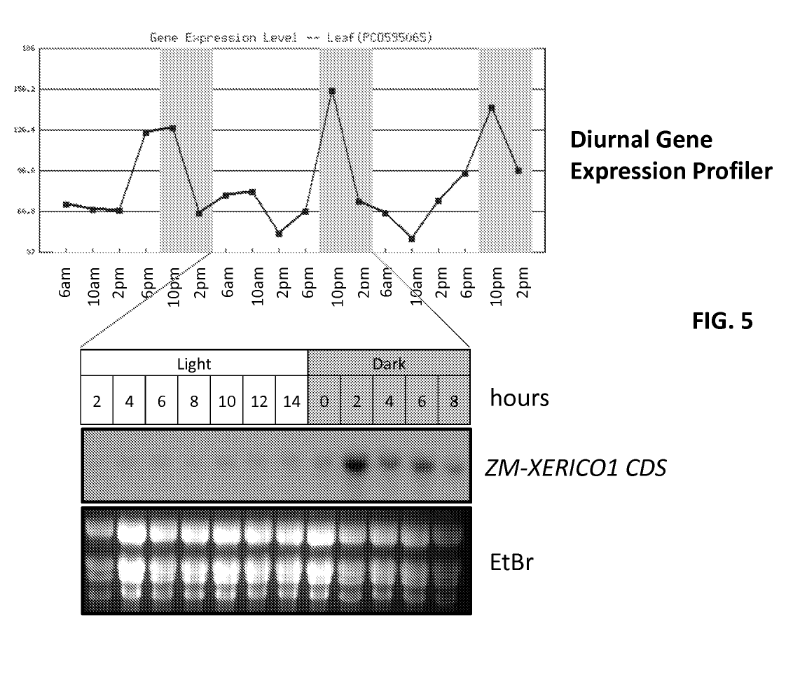

Expression of ZmXERICO1 and 2 was assayed for V4-V5 B73 seedlings. Seedlings were subjected to water withdrawal for 48 h (hours) and rewatered thereafter. Shoot and root samples were collected before water stress, at 24 and 48 h after water stress and 24 h after rewatering. Northern blot analysis using molecular probes specific to each ZmXERICO gene indicates that ZmXERICO1 and 2 are both expressed in root tissue whereas only ZmXERICO1 appears highly expressed in shoots. Expression of ZmXERICO1 was highly inducible in shoots and roots whereas ZmXERICO2 was induced by drought stress in roots to a lesser extent. (See, FIG. 4). This apparent organ specificity of induction is consistent in the context of a possible role for Xerico in increasing ABA levels to control stomatal aperture under stress.

The data also demonstrate that drought-induced expression of ZmXERICO genes in maize plants lessens when water supply returns to adequate levels.

Over-Expression of ZmXERICO1 and ZmXERICO2

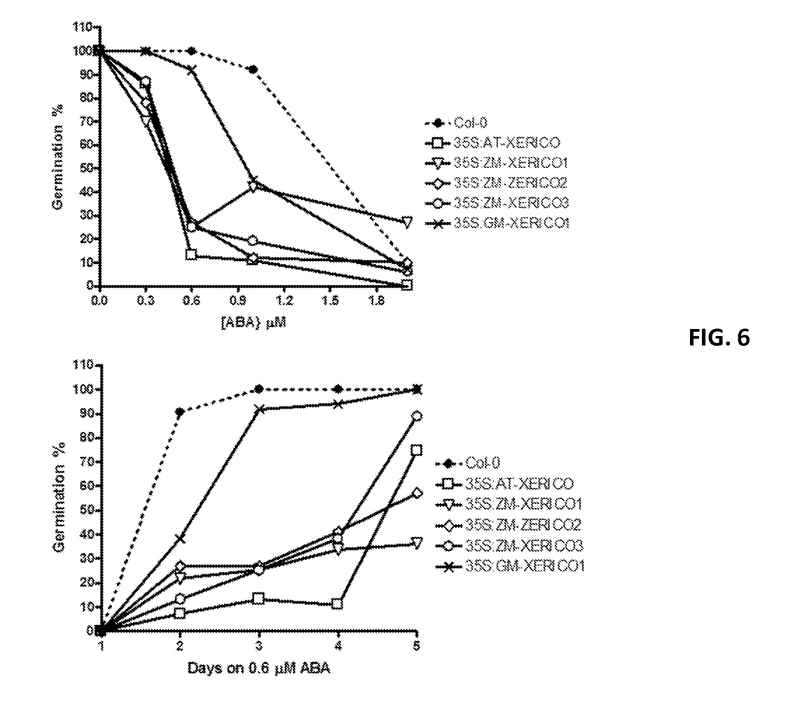

Arabidopsis Columbia-0 wild-type plants were transformed with a construct aimed at over-expressing ZmXERICO1, ZmXERICO2, ZmXERICO1A, AtXERICO or GmXERICO1. FIG. 6 shows an increased ABA sensitivity of ZmXERICO1, 2 and 1A compared to controls and GmXERICO1 as measured by germination percentage on MS (Murashige and Skoog) plates containing different ABA concentrations after 3 days. A marked difference could be seen at 0.6 μM, except for GM-XERICO1 transgenic, indicating that this gene is likely not active or is less active than maize and Arabidopsis genes. FIG. 6 shows the evolution of germination for different transgenic Arabidopsis plants compared to controls, demonstrating the increased ABA sensitivity of ZmXERICO1, 2 and 1A transgenic plants compared to controls. Transgenic corn plants were produced to over-express ZmXERICO1 or ZmXERICO2.

Expressing events could be identified from the aerial view and showed signs of tolerance to drought on the ground. In particular, transgenic plants showed visibly healthier canopies under stress, delayed firing of lower leaves and little leaf-rolling, as well as less tassel blasting compared to non-transgenic controls. The only event not showing a visible difference compared to controls was the non-expressing ZmXERICO1 event.

Another interesting phenotype is the apparent faster drying time and senescence of husk leaves on ZmXERICO1 events. Expression optimization, for example by using a promoter expressed in leaves but not in ear or husk leaves, could alter this phenotype.

As shown in Table 2, transgenic ZmXERICO1 corn plants in the field appear able to produce at least one ear, and ASI seems to be similar or reduced compared to bulk nulls (BN) depending on the event considered. An exception is event #5. (WO ASI, anthesis-silking interval measured in managed-stressed environment (WO); STAGRN, staygreen phenotype measured in WO in plot subjected to a flowering stress (FS) or a grain filling stress (GFS)) The staygreen phenotype was quantified on a scale from 1 to 9 and is indicative of a significantly healthier canopy for expressing transgenic events compared to control or a non-expressing event (Event #8). These data indicate that overexpression of a ZmXERICO gene enhances drought tolerance in transgenic plants compared to controls.

TABLE 2
ASI and Number of Plants Without Ears in Transgenic and control plots

| | | # of | | | |
|---|---|---|---|---|---|
| | | plants | | | |
| | Event | w/o | WO | WO_FS | WO_GFS |
| Entry_Comment | name | ears | ASI | (STAGRN) | (STAGRN) |
| UBI:ZM- | Event #1 | 2.3 | 4 | 7.7 | 7.8 |
| XERICO1 | Event #2 | 4 | 19 | 7.3 | 8.1 |
| | Event #3 | 3 | 6 | 7.0 | 7.8 |
| | Event #4 | 2.3 | 3 | 7.1 | 7.5 |
| | Event #5 | 11 | 51 | 7.2 | 8.1 |
| | Event #6 | 4.7 | 11 | 7.1 | 8.1 |
| | Event #7 | 8 | 23 | 7.1 | 8.1 |
| | Event #8 | 7.7 | 34 | 5.5 | 6.1 |
| | Event #9 | 7 | 1 | 7.6 | 8.5 |
| | Event #10 | 5 | 17 | 7.3 | 8.1 |
| | BN | 10.3 | 38 | 5.3 | 6.1 |

### Example 2

### Analysis of ABA Levels in Transgenic and Control Plants

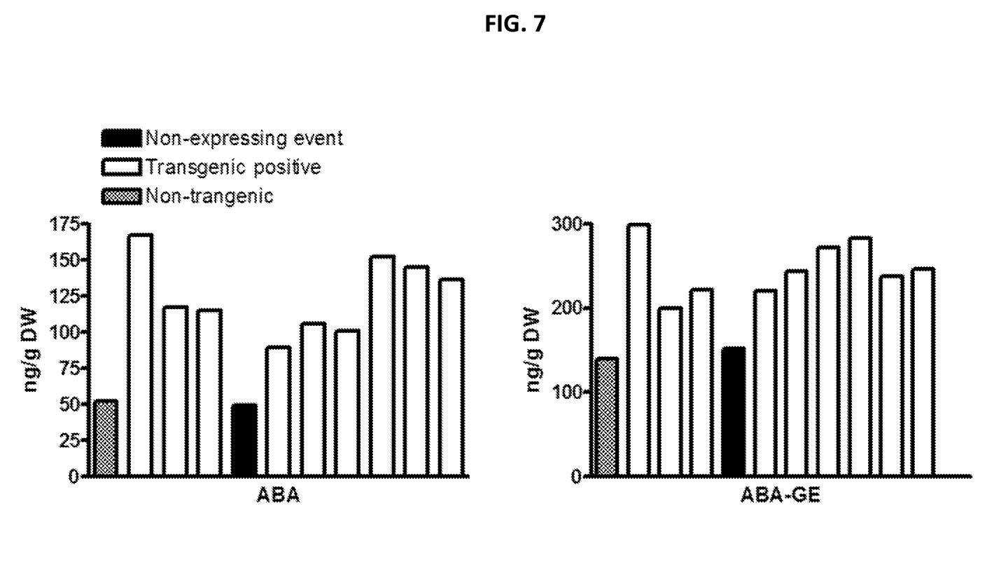

An analysis of ABA levels in transgenic and control plants was. Samples (no replication) were collected in the field under well watered conditions. Results indicated that both ABA and ABA-GE levels are up in expressing events compared to bulk null control or non-expressing event. ABA levels were increased by an average of 2-3 times whereas ABA-GE levels were increased 1.7 times on average (FIG. 7). However, the increase in ABA and ABA-GE levels observed in transgenic ZmXerico expressing plants was within the biologically relevant levels seen in stressed non-transgenic control plants.

In order to further study the differences in ABA and ABA derivatives in transgenic maize plants over-expressing ZmXERICO1 or ZmXERICO2 compared to controls, leaf and immature ear material were collected from plants grown under well-watered (WW) or water stressed condition before flowering (FS). Samples were immediately plunged in liquid nitrogen and stored at −80° C. Frozen tissue was ground in liquid nitrogen and lyophilized. Hormone analysis was carried out as previously described (Chiwocha, et al., (2003) Plant Journal 10:1-13). Analysis of the data demonstrates that Ubi::ZmXERICO1 transgenic plants have higher leaf levels of ABA, ABA-GE and 7′-OH ABA but lower leaf levels of ABA's two metabolites: phaseic acid (PA) and dihydrophaseic acid (DPA)). Similarly, it was observed that Ubi::ZmXERICO2 transgenic plants had higher leaf levels of ABA, ABA-GE, and 7′-OH ABA but lower leaf levels of PA and DPA).

To assess hormone levels in reproductive tissues, hormone profiling assays were repeated in Ubi:: ZmXERICO1 transgenic plants using immature ear collected prior to silking. Immature ears were cut in half generating immature ear “tip” and “base” samples. The data indicated that these transgenic corn plants have higher immature ear levels of ABA, ABA-GE, and 7′-OH ABA, but lower levels of PA and DPA in immature ears, especially under drought stress conditions.

Hormone profiling experiments were performed to determine the levels of ABA, ABA-GE, DPA, PA, and 7′-OH ABA in ZmXERICO1 and 2 transgenic and control plants under well watered and flowering stress conditions for various tissue type—leaf, immature ear-base and immature ear-tip. In summary, ABA levels were 2.9 fold higher in the transgenic plants compared to the bulk-null control plants under flowering stress. Under well watered conditions, transgenic plants had 4.5 times higher ABA levels than bulk-null control plants. This increase was consistent across the tissue types tested e.g., leaf, immature ear-base and immature ear-tip. Similarly, ABA-GE levels were 2.3 fold higher in the transgenic plants compared to the bulk-null control plants under flowering stress. Under well watered conditions, transgenic plants had 2.8 times higher ABA-GE levels than bulk-null control plants. This increase was consistent across the tissue types tested e.g., leaf, immature ear-base and immature ear-tip.

However, DPA levels were 1.5 fold lower in transgenic plants compared to the bulk-null control plants under flowering stress. Under well watered conditions, DPA levels were also 1.5 fold lower in transgenic plants than in bulk-null control plants. This observation was consistent across the tissue types tested e.g., leaf, immature ear-base and immature ear-tip. Similarly, PA levels were 2.8 fold lower in transgenic plants compared to the bulk-null control plants under flowering stress. Under well watered conditions, DPA was undetectable in transgenics compared to controls. This observation was consistent across the tissue types tested e.g., leaf, immature ear-base and immature ear-tip.

Thus, an increase in leaf ABA metabolites mentioned above is accompanied by a reduction in phaseic acid (PA) and diphaseic acid (DPA) levels in transgenic plants compared to controls. ZmXERICO modulates levels of ABA metabolites through a decrease in ABA degradation and not an increase in ABA biosynthesis. If the second conjecture were true, PA and DPA levels would also be increased in transgenic leaf tissues. The data presented here indicate that ZmXERICO genes are negative regulators of ABA degradation, rather than positive regulators of ABA biosynthesis as suggested by others. Therefore, ZmXERICO appears to reduce endogenous ABA degradation by acting as a negative regulator and does not increase the biosynthesis of endogenous ABA.

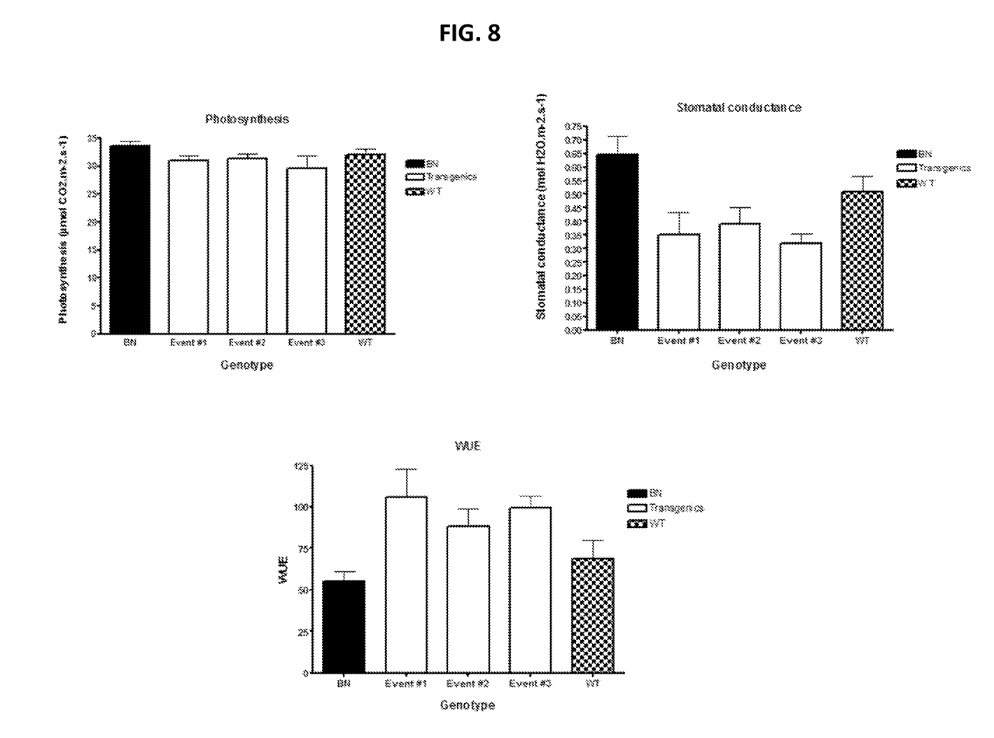

FIG. 8 shows results of carbon exchange rate (CER, photosynthesis) and stomatal conductance (a measure of leaf air/water exchange through stomates) measurements in transgenic and WT and bulk null controls grown in the greenhouse under normal conditions. Data shows that transgenic plants have higher water use efficiency (WUE) (calculated as Photosyntheis/Stomatal conductance) than control plants, indicating that ZmXERICO1 trangenics' evapo-transpiration rate is reduced without significant impact on CER, likely because of the increase in ABA levels described above.

The main pathway of ABA degradation is catalyzed by ABA 8′-hydroxilases (also known as ABA 8′-oxidases). See, Kushiro, et al., (2004) EMBO J 23:1647-1656. The enzymes are cytochrome P450 proteins (CYP707A) that catalyze the 8′-hydroxylation of ABA. This in turn leads to the production of PA that is converted into DPA. PA and DPA do not have ABA-like activity and are therefore considered inactive. In yeast and mammals, the activity of some cytochrome P450s is regulated at the posttranslational level through Endoplasmic Reticulum-associated degradation (ERAD). ERAD constitutes (1) the ubiquitination of the P450 target and (2) the degradation of the ubiquitinated proteins by the 26S proteasome. This ubiquitination process requires an E3-ubiquitin ligase. Proteins containing RING-H2 domains have often been shown to have E3-ubiquitin ligase activity and ZmXERICO proteins are predicted to be targeted to the ER and they each have a putative transmembrane domain. ZmXERICO proteins, and possibly other related RING-H2s, appear to play a role in the regulation of ABA 8′-hydroxylases ERAD in corn.

Specifically, it is hypothesized that ZmXERICO may function as an E3-Ubiquitin ligase to regulate degradation of ER-anchored P450 ABA 8′-hydroxylases

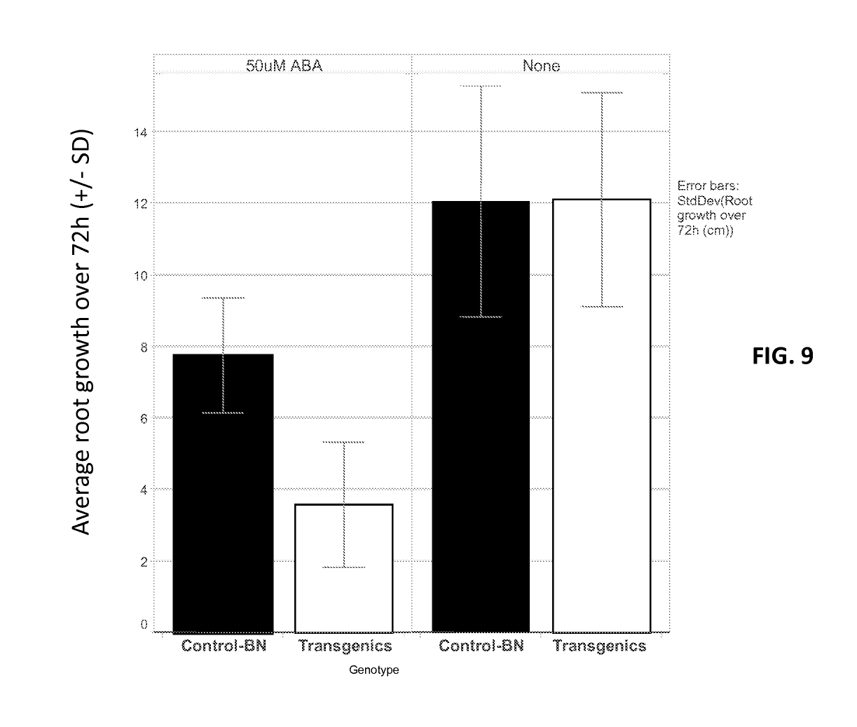

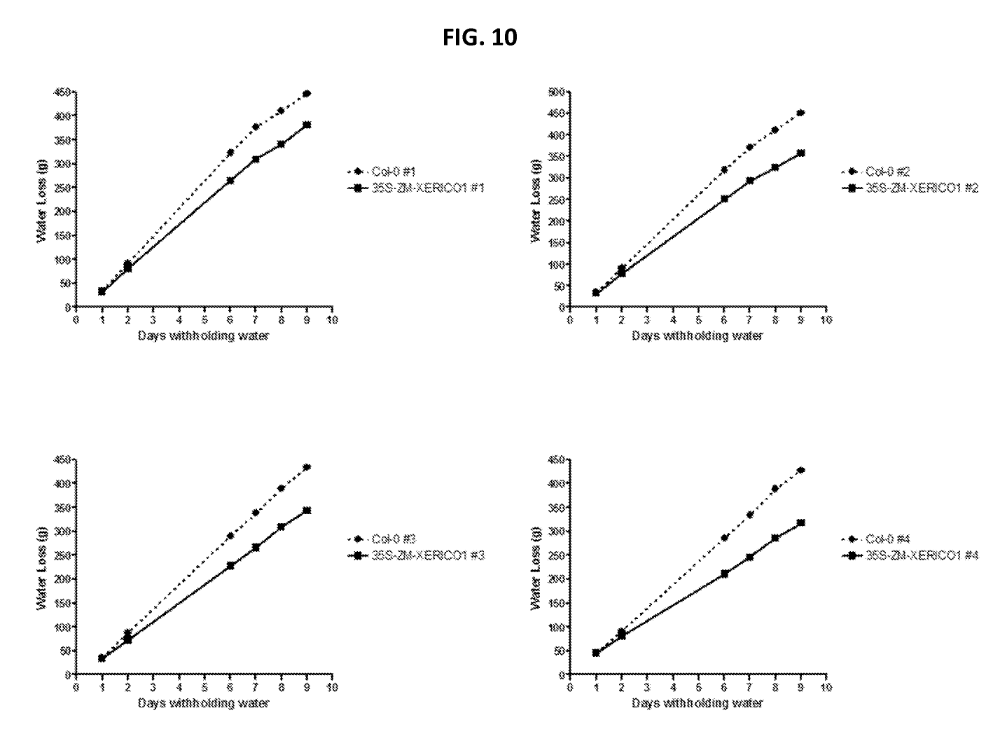

It was found that transgenic maize seedlings over-expressing ZmXERICO1 were hypersensitive to ABA compared to controls as demonstrated by the measure of root growth rate in germ paper soaked with 50 uM ABA over 72 h. No root growth rate difference was found without ABA treatment (FIG. 9).

### Example 3

### Drought Tolerance Screening of Transgenic Plants Expressing XERICO Proteins

A qualitative drought screen was performed with plants over-expressing different Xerico genes under the control of different promoters. The soil is watered to saturation and then plants are grown under standard conditions (i.e., 16 hour light, 8 hour dark cycle; 22° C.; ˜60% relative humidity). No additional water is given. Digital images of the plants are taken at the onset of visible drought stress symptoms. Images are taken once a day (at the same time of day), until the plants appear dessicated. Typically, four consecutive days of data is captured.

Color analysis is employed for identifying potential drought tolerant lines. Maintenance of leaf area is also used as another criterion for identifying potential drought tolerant lines, since Arabidopsis leaves wilt during drought stress. Maintenance of leaf area can be measured as reduction of rosette leaf area over time.

The four-day interval with maximal wilting is obtained by selecting the interval that corresponds to the maximum difference in plant growth. The individual wilting responses of the transgenic and wild-type plants are obtained by normalization of the data using the value of the green pixel count of the first day in the interval. The drought tolerance of the transgenic plant compared to the wild-type plant is scored by summing the weighted difference between the wilting response of activation-tagged plants and wild-type plants over day two to day four; the weights are estimated by propagating the error in the data. A positive drought tolerance score corresponds to a transgenic plant with slower wilting compared to the wild-type plant. Significance of the difference in wilting response between activation-tagged and wild-type plants is obtained from the weighted sum of the squared deviations. Lines with a significant delay in yellow color accumulation and/or with significant maintenance of rosette leaf area, when the transgenic replicates show a significant difference (score of greater than 0.9) from the control replicates, the line is then considered a validated drought tolerant line.

Using the assay described herein, plants with a Drought tolerance score of greater than 0.9 and a positive Deviation identify plants are considered significantly more drought tolerant than controls. Arabidopsis seedlings overexpressing ZMXERICO1, ZMXERICO2 and ZMXERICO1A under the control of the 35S promoter had particularly high scores for drought tolerance. Scores obtained with ZmXERICO genes were higher than the score obtained with Arabidopsis Xerico gene. In addition, transgenic plants expressing ZmXERICO1 under the control of a root specific promoter (RSP) also showed significantly higher drought tolerance compared to control plants. The results indicate that ZmXERICO genes can be used under the control of different promoters to improve drought tolerance in transgenic Arabidopsis plants.

TABLE 3
Drought tolerance scores for Arabidopsis seedlings expressing
ZMXERICO1 or ZMXERICO2.

| | | Drought | |
|---|---|---|---|
| | | tolerance | |
| Promoter | Gene | score (2 sigma) | Deviation |
| 35S | At2g04240 | 3.786 | 12.887 |
| RAB18 | At2g04240 | 0.832 | 0.417 |
| RD29A | At2g04240 | 0.243 | −0.474 |
| RSP | At2g04240 | 1.422 | −2.399 |
| 35S | GM-XERICO | 0.682 | 1.87 |
| 35S | ZM-XERICO1 | 6.18 | 21.251 |
| RAB18 | ZM-XERICO1 | 0.352 | −0.601 |
| RSP | ZM-XERICO1 | 2.087 | 6.965 |
| 35S | ZM-XERICO2 | 6.259 | 18.718 |
| 35S | ZM-XERICO1A | 4.842 | 15.41 |

Bold and underlined entries indicate statistically significant differences compared to the control plants.

### Example 4

### Transformation and Regeneration of Transgenic Plants

Immature maize embryos from greenhouse donor plants are bombarded with a plasmid containing the Xerico gene operably linked to a promoter and the selectable marker gene PAT (Wohlleben, et al., (1988) Gene 70:25-37), which confers resistance to the herbicide bialaphos. Alternatively, the selectable marker gene is provided on a separate plasmid. Transformation is performed as follows. Media recipes follow below.

### Preparation of Target Tissue

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising a ZmXERICO gene operably linked to a promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288 J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for ABA levels and/or drought tolerance.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288 J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288 J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 5

### Agrobacterium-mediated Transformation

For Agrobacterium-mediated transformation of maize with a Xerico polynucleotide sequence of the invention, the method of Zhao is employed (U.S. Pat. No. 5,981,840, and PCT Patent Publication Number WO 1998/32326; the contents of which are hereby incorporated by reference; see, also, Zhao, et al., (1998) Maize Genetics Cooperation Newsletter 72:34-37). Briefly, immature embryos are isolated from maize and the embryos contacted with a suspension of Agrobacterium, where the bacteria are capable of transferring the Xerico polynucleotide of interest to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### Example 6

### Soybean Embryo Transformation

### Culture Conditions

Soybean embryogenic suspension cultures (cv. Jack) are maintained in 35 ml liquid medium SB196 (see, recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein, et al., (1987) Nature 327:70).

### Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed is cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

### Preparation of DNA for Bombardment

Either an intact plasmid or a DNA plasmid fragment containing the genes of interest and the selectable marker gene are used for bombardment. Plasmid DNA for bombardment are routinely prepared and purified using the method described in the Promega™ Protocols and Applications Guide, Second Edition (page 106). Fragments of the plasmids carrying the Xerico polynucleotide of interest are obtained by gel isolation of double digested plasmids. In each case, 100 ug of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing Xerico polynucleotide of interest are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e. per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

### Selection of Transformed Embryos

Transformed embryos were selected either using hygromycin (when the hygromycin phosphotransferase, HPT, gene was used as the selectable marker) or chlorsulfuron (when the acetolactate synthase, ALS, gene was used as the selectable marker).

### Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

### Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos into Plants

In order to obtain whole plants from embryogenic suspension cultures, the tissue must be regenerated.

### Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 μE/m²s. After this time embryo clusters are removed to a solid agar media, SB166, for 1-2 weeks. Clusters are then subcultured to medium SB103 for 3 weeks. During this period, individual embryos can be removed from the clusters and screened for ABA accumulation. It should be noted that any detectable phenotype, resulting from the expression of the genes of interest, could be screened at this stage.

### Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins.

### Media Recipes

SB 196 - FN Lite liquid proliferation medium (per liter) -

| | | |
|---|---|---|
| MS FeEDTA - 100x Stock 1 | 10 | ml |
| MS Sulfate - 100x Stock 2 | 10 | ml |
| FN Lite Halides - 100x Stock 3 | 10 | ml |
| FN Lite P, B, Mo - 100x Stock 4 | 10 | ml |
| B5 vitamins (1 ml/L) | 1.0 | ml |
| 2,4-D (10 mg/L final concentration) | 1.0 | ml |
| KNO3 | 2.83 | gm |
| (NH4)2SO4 | 0.463 | gm |
| Asparagine | 1.0 | gm |
| Sucrose (1%) | 10 | gm |
| pH 5.8 | | |

FN Lite Stock Solutions

| Stock # | | 1000 ml | | 500 ml | |
|---|---|---|---|---|---|
| 1 | MS Fe EDTA 100x Stock | | | | |
| | Na₂ EDTA* | 3.724 | g | 1.862 | g |
| | FeSO₄—7H₂O | 2.784 | g | 1.392 | g |

*Add first, dissolve in dark bottle while stirring

| | | | | | |
|---|---|---|---|---|---|
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | Kl | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

SB1 solid medium (per liter) comprises: 1 pkg. MS salts (Gibco/BRL—Cat#11117-066); 1 ml B5 vitamins 1000× stock; 31.5 g sucrose; 2 ml 2,4-D (20 mg/L final concentration); pH 5.7; and, 8 g TC agar.

SB 166 solid medium (per liter) comprises: 1 pkg. MS salts (Gibco/BRL—Cat#11117-066); 1 ml B5 vitamins 1000× stock; 60 g maltose; 750 mg MgCl2 hexahydrate; 5 g activated charcoal; pH 5.7; and, 2 g gelrite.

SB 103 solid medium (per liter) comprises: 1 pkg. MS salts (Gibco/BRL—Cat#11117-066); 1 ml B5 vitamins 1000× stock; 60 g maltose; 750 mg MgCl2 hexahydrate; pH 5.7; and, 2 g gelrite.

SB 71-4 solid medium (per liter) comprises: 1 bottle Gamborg's B5 salts w/ sucrose (Gibco/BRL—Cat#21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20 C comprises: 10 g myo-inositol; 100 mg nicotinic acid; 100 mg pyridoxine HCl; and, 1 g thiamine. If the solution does not dissolve quickly enough, apply a low level of heat via the hot stir plate. Chlorsulfuron Stock comprises 1 mg/ml in 0.01 N Ammonium Hydroxide.

### Example 7

### Sunflower Meristem Tissue Transformation

Sunflower meristem tissues are transformed with an expression cassette containing the Xerico polynucleotide operably linked to a promoter as follows (see also, European Patent Number EP 0 486233, herein incorporated by reference, and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant. 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6, and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the Xerico gene operably linked to the promoter is introduced into Agrobacterium strain EHA105 via freeze-thawing as described by Holsters, et al., (1978) Mol. Gen. Genet. 163:181-187. The plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for Xerico activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by Xerico activity analysis of leaf extracts while transgenic seeds harvested from NPTII-positive T₀ plants are identified by Xerico activity analysis of small portions of dry seed cotyledon.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method for increasing drought tolerance in a plant, said method comprising:
a) expressing a recombinant nucleotide sequence encoding a polypeptide having at least 90% sequence identity to SEQ ID NO: 2 (ZmXERICO1), SEQ ID NO:m4 (ZmXERICO2), or SEQ ID NO: 6 (ZmXERICO1A), wherein said nucleotide sequence is operably linked to a heterologous promoter selected from the group consisting of a weak constitutive promoter, an organ- or tissue-preferred promoter a stress-inducible promoter, a chemical-induced promoter, a light-responsive promoter, and a diurnally-regulated promoter; and
b) expressing said nucleotide sequence in said plant;
whereby drought tolerance of said plant is increased relative to a control plant.

2. The method of claim 1, wherein said weak constitutive promoter is a GOS2 promoter or rice actin promoter.

3. The method of claim 1, wherein said organ- or tissue-preferred promoter is a leaf-preferred promoter, a root-preferred promoter, a vasculature-specific promoter or a promoter without expression in developing or mature ears.

4. The method of claim 1, wherein said stress-inducible promoter is a Rab17 promoter or an Rd29a promoter.

5. The method of claim 1, wherein said light-responsive promoter is an rbcS (ribulose-1,5-bisphosphate carboxylase) promoter, a Cab (chlorophyll a/b-binding) promoter or a phosphoenol-pyruvate carboxylase (PEPc) promoter.

6. The method of claim 1, wherein said diurnally-regulated promoter is disclosed in PCT/US2011/020314.

7. A method for increasing yield of a seed crop plant exposed to drought stress, said method comprising increasing expression of a polypeptide having at least 90% sequence identity to SEQ ID NO: 2, 4 or 6 in said plant and resulting in changed abscisic acid (ABA) homeostasis levels and/or decreasing responsiveness of developing seed of said plant to ABA.

8. The method of claim 7, wherein said crop plant further comprises an ABA-associated sequence operably linked to a heterologous promoter that drives expression in developing seed tissues.

9. The method of claim 8, wherein said ABA-associated sequence encodes an ABA-insensitive ABI mutant.

10. The method of claim 9, wherein said ABA-insensitive ABI mutant is selected from the group consisting of abi1, abi2, and ZmABI1 mutant.

11. The method of claim 7, wherein said seed crop plant is selected from the group consisting of a grain plant, an oil-seed plant, and a leguminous plant.

12. The method of claim 11, wherein said grain plant is corn or wheat.

13. The method of claim 11, wherein said oil-seed plant is a Brassica plant.

14. The method of claim 8, wherein said promoter is an early kernel/embryo promoter.

15. The method of 7, wherein the rate of degradation of ABA is decreased.

16. A plant comprising a polynucleotide construct comprising a nucleotide sequence encoding a polypeptide having at least 90% sequence identity to SEQ ID NO: 2, SEQ ID NO: 4, or SEQ ID NO: 6, wherein said nucleotide sequence is operably linked to a heterologous promoter selected from the group consisting of a weak constitutive promoter, an organ- or tissue-preferred promoter, a stress-inducible promoter, a chemical-induced promoter, a light-responsive promoter, and a diurnally-regulated promoter and wherein the plant exhibits increased drought tolerance relative to a control.

17. The plant of claim 16, wherein said polynucleotide is stably incorporated into the genome of said plant.

18. The plant of claim 16, wherein said plant is a seed crop plant.

19. (canceled)

20. The method of claim 3, wherein said root-preferred promoter is maize Cyclo1, maize RootMET2, or sorghum Rcc3.

21. A method of improving drought tolerance in a population of crop plants, the method comprising (a) expressing a recombinant protein comprising RING-H2 zinc finger motif, wherein the RING-H2 domain is present in one of SEQ ID NO: 2, SEQ ID NO: 4 or SEQ ID NO: 6; (b) exposing the crop plants to a drought condition in a field; and (c) improving the drought tolerance of the population of crop plants in the field.

22. A method of reducing phaseic acid (PA) and dihydrophaseic acid (DPA) levels in a plant, the method comprising (a) expressing a recombinant protein comprising RING-H2 zinc finger motif, wherein the RING-H2 domain is present in one of SEQ ID NO: 2, SEQ ID NO: 4 or SEQ ID NO: 6; (b) exposing the crop plants to a drought condition in a field; and (c) reducing the phaseic acid (PA) and dihydrophaseic acid (DPA) levels in plant, while increasing the levels of ABA in the plant.


SEQUENCE LISTING (10 sequences)

SEQ ID NO: 1 - DNA, 474 bp, Zea mays
atggggatctcgagcatgccggagccgcgggacagcctgctggggtacctggtgtacaacacggtgatctcgatcgcggcgctggcggggctggtgcgggcggcgctggtgttcctggacctgcaggccgcgctgccgcccggggacgacggcggggaccgactggcggcgtcggcgcccggcctggcggagcggttcctcagggccttccggccggcgctgtacgaggtgctggcgtcgacggcgacgacgtgcgaggcggacggcggcggcggcggcgacgactgcagcgtgtgcctggccgggttccgggcgagggccgtggtgaaccgcctcccctgcggccacctcttccaccgcgcctgcctcgagacctggctccggtacgagcgcgccacgtgcccgctctgccgcgcccacgtgcccctccccgccgacgagacgccgctgctccgctacccggagttcgagtga

SEQ ID NO: 2 - protein, 157 aa, Zea mays
MGISSMPEPRDSLLGYLVYNTVISIAALAGLVRAALVFLDLQAALPPGDDGGDRLAASAPGLAERFLRAFRPALYEVLASTATTCEADGGGGGDDCSVCLAGFRARAVVNRLPCGHLFHRACLETWLRYERATCPLCRAHVPLPADETPLLRYPEFE

SEQ ID NO: 3 - DNA, 498 bp, Zea mays
atggggatctcgagcatgccggagccgcgggacagcctgctggggtacctggtgtacaacgcggtggtgtcgatcgcggcgctggcggggctggtgcgggcggcgctggtgttcctggacctgcaggccgcgcagctgcccgggggcgcgggcgcgggtgcggacgacgacggcggggaccgtctcgcggcgtcgggcccgggcctgggcctggcggagcggttcctgagggccttccggccggcgctgtacggggtgctggtgtccacggcgtgcggcgcggcggaggcggccgcgggcgacgacgactgcagcgtgtgcctggccgggttcgaggcggaggccgtggtgaaccggctcccctgcggccacctcttccaccgcgcctgcctcgagacctggctccggtacgagcgcgccacgtgcccgctctgccgcgcccacgtgcccctccccgccgacgagacgccggtgctccgctacccggagctcgagtga

SEQ ID NO: 4 - protein, 165 aa, Zea mays
MGISSMPEPRDSLLGYLVYNAVVSIAALAGLVRAALVFLDLQAAQLPGGAGAGADDDGGDRLAASGPGLGLAERFLRAFRPALYGVLVSTACGAAEAAAGDDDCSVCLAGFEAEAVVNRLPCGHLFHRACLETWLRYERATCPLCRAHVPLPADETPVLRYPELE

SEQ ID NO: 5 - DNA, 465 bp, Zea mays
atggggatctcgagcatgccggagccgcgggacagcctgctggggtacctggtgtacaacacggtgatctcgatcgcggcgctggcggggctggtgcgggcggcgctggtgttcctggacctgcaggccgcgctgccgcccggggacgacggcggggaccaactggcggcgtcgggcccgggactggcagagcggttcctcagggccttccggccggcgctgtacgaggtgctggcgtcgacggcgacgacgtgcgaggcggacggcggcggcgacgactgcagcgtgtgcctggccgggttccgggcgagggccgtggtgaaccgcctcccctgcggccacctcttccaccgcgcctgcctcgagacctggctccggtacgagcgcgccacgtgcccgctctgccgcgcccacgtgcccctccccgccgacgagacgccgctgctccgctacccggagttcgag

SEQ ID NO: 6 - protein, 155 aa, Zea mays
MGISSMPEPRDSLLGYLVYNTVISIAALAGLVRAALVFLDLQAALPPGDDGGDQLAASGPGLAERFLRAFRPALYEVLASTATTCEADGGGDDCSVCLAGFRARAVVNRLPCGHLFHRACLETWLRYERATCPLCRAHVPLPADETPLLRYPEFE

SEQ ID NO: 7 - protein, 167 aa, Artificial
  Consensus sequence of Figure 1
MGISSMPEPRDSLLGYLVYNTVISIAALAGLVRAALVFLDLQAAXXPGXXXAXXXDDGGDRLAASAPXXLGLAERFLRAFRPALYXVLXSTACXCXXXAAGXXDDCSVCLAGFXADAVVNRLPCGHLFHRACLETWLRYERATCPLCRAHVPLPADETPLLRYPEXE

SEQ ID NO: 8 - DNA, 1407 bp, Zea mays
atggaagacgtcgtagcagtcgtggcgtcactctccgcgccgccggcgccggcgtttagccccgccgcggtggtcgtcggagccatggagggggtctccgtgcccgtgactgtgcccccggtcaggacggcgtccgcggtggacgacgacgcgctggcgccgggagaggaagggggagacgcctctttggccgggagcccgtgctcggtggtcagcgactgtagcagcgtggccagcgctgatttcgagggggtcgggctgtgtttcttcggcgcggcagcaggcgcggagggtggtcccatggtgttggaggactcgaccgcgtctgcagccacggtcgaggcggaggccagggtcgcggctggtgggaggagtgtcttcgccgtggactgcgtgccgctgtggggctacacttccatatgcggccgccgtccggagatggaggatgccgttgctatagtgccgcgattctttgacttgccactctggttgctcaccggcaatgcgatggtcgatggcctcgatcccatgacgttccgcttacctgcacatttctttggtgtctatgacggacacgatggtgcacaggtagcaaattactgtcgggaacgcctccatgtggccctactggagcagctgagcaggatagaggagactgcgtgtgcagctaacttgggagacatggagttcaagaaacagtgggaaaaggtctttgtggattcttatgctagagtggatgacgaggttgggggaaacacgatgaggggaggtggtgaagaagcaggcacaagtgatgctgctatgacactcgtgccagaacctgtggcacctgagacggtgggttcgacggcggtcgtcgctgtcatctgctcctcacatatcattgtctccaactgtggagattcacgggcagtgctctgccgaggcaagcagcctgtgcctctgtcggtggatcataaacctaacagggaggatgagtatgcaaggattgaggcagagggtggcaaggtcatacaatggaacggttatcgagttttcggtgttcttgcaatgtcgcgatcaattggtgacagatatctgaagccatggataattccagtcccagaggtaacaatagttccgcgggctaaggatgacgagtgccttattcttgccagtgacggcctctgggatgtaatgtcaaatgaagaggtatgtgaaatcgctcgcaagcggatacttctgtggcacaaaaagaacagcacaagctcatcatcagccccacgggttggtgactccgcagactcagccgctcaagcggctgctgaatgcttgtcaaagcttgctcttcagaaggggagcaaagacaacattactgtcgtggtagttgatctgaaagcacagcgcaagttcaagagcaaaacttaa

SEQ ID NO: 9 - protein, 468 aa, Zea mays
MEDVVAVVASLSAPPAPAFSPAAVVVGAMEGVSVPVTVPPVRTASAVDDDALAPGEEGGDASLAGSPCSVVSDCSSVASADFEGVGLCFFGAAAGAEGGPMVLEDSTASAATVEAEARVAAGGRSVFAVDCVPLWGYTSICGRRPEMEDAVAIVPRFFDLPLWLLTGNAMVDGLDPMTFRLPAHFFGVYDGHDGAQVANYCRERLHVALLEQLSRIEETACAANLGDMEFKKQWEKVFVDSYARVDDEVGGNTMRGGGEEAGTSDAAMTLVPEPVAPETVGSTAVVAVICSSHIIVSNCGDSRAVLCRGKQPVPLSVDHKPNREDEYARIEAEGGKVIQWNGYRVFGVLAMSRSIGDRYLKPWIIPVPEVTIVPRAKDDECLILASDGLWDVMSNEEVCEIARKRILLWHKKNSTSSSSAPRVGDSADSAAQAAAECLSKLALQKGSKDNITVVVVDLKAQRKFKSKT

SEQ ID NO: 10 - protein, 162 aa, Arabidopsis thaliana
MGLSSLPGPSEGMLCVILVNTALSISIVKGIVRSFLGIVGISLSPSSSSPSSVTVSSENSSTSESFDFRVCQPESYLEEFRNRTPTLRFESLCRCKKQADNECSVCLSKFQGDSEINKLKCGHLFHKTCLEKWIDYWNITCPLCRTPLVVVPEDHQLSSNVW